SEQUENCE LISTING (3 sequences)

SEQ ID NO: 1 - DNA, 82 bp, synthetic construct
  misc_feature 1..82: note = Synthetic Sequence
  misc_feature 1: note = terminal 5' phosphate
  source 1..82: mol_type = other DNA
cgcgtcgctcgtcggcagcgtcagatgtgtataagagacagcgcgtcgctcgtcggcagcgtcagatgtgtataagagacag

SEQ ID NO: 2 - DNA, 39 bp, synthetic construct
  misc_feature 1..39: note = Synthetic Sequence
  source 1..39: mol_type = other DNA
acgacgctcttccgatctgagttctacagtccgacgatc

SEQ ID NO: 3 - DNA, 50 bp, synthetic construct
  misc_feature 1..50: note = Synthetic Sequence
  modified_base 23: mod_base = OTHER; note = 5-Nitroindole
  misc_difference 23: note = n is a, c, g, t, or 5-Nitroindole
  misc_feature 50: note = terminal 3' Hexanediol
  source 1..50: mol_type = other DNA
gacgctgccgacgagcgacgcgngatcgtcggactgtagaactcagatcg

(19) United States
(12) Patent Application Publication
(10) Pub. No.: US 2023/0235385 A1
(43) Pub. Date: Jul. 27, 2023

(54) METHOD FOR ADDING SPATIALLY-ADDRESSABLE BARCODES TO NUCLEIC ACIDS OF A CELLULAR SAMPLE IN SITU

(71) Applicant: Scale Biosciences Inc., San Diego, CA (US)

(72) Inventors: Frank STEEMERS, San Diego, CA (US); Garry P. NOLAN, Redwood City, CA (US)

(21) Appl. No.: 18/181,492

(22) Filed: Mar. 9, 2023

Related U.S. Application Data
(63) Continuation of application No. PCT/US2022/019382, filed on Mar. 8, 2022.
(60) Provisional application No. 63/161,834, filed on Mar. 16, 2021.

(51) Int. Cl. C12Q 1/6841; C12Q 1/6869; C12Q 1/6876; C12Q 1/686

(52) U.S. Cl. CPC C12Q 1/6841; C12Q 1/6869; C12Q 1/6876; C12Q 1/686; C12Q 2600/16

(57) ABSTRACT

Provided herein, among other things, is a method for synthesizing spatially addressed nucleic acid barcodes in or on a cellular sample in situ. In some embodiments, the method may comprise: obtaining a cellular sample comprising nucleic acid molecules that are protected by a reversible terminator, deprotecting the nucleic acid molecules in a set of areas of the sample by selectively applying an external stimulus to the set of areas to produce deprotected nucleic acid molecules in the areas, applying a reversible terminator nucleotide to the cellular sample, resulting in addition of a reversible terminator onto the deprotected nucleic acid molecules, optionally removing any unreacted reversible terminator nucleotide from the sample, and repeating the steps one or more times.

### CROSS-REFERENCING

This application claims the benefit of U.S. provisional application Ser. No. 63/161,834, filed on Mar. 16, 2021, which application is incorporated by reference for all purposes.

### BACKGROUND

Gene expression analysis has become a standard tool for studying how genes are regulated, cellular states and cellular functions. However, transcription in individual cells is influenced by their localization within a particular tissue. As such, to gain a more complete understanding of a cell, one should obtain information about gene expression in individual cells in their morphological context.

Current methods for analyzing gene expression in tissue sections are limited. For example, in situ hybridization provides a way to analyze transcripts in a tissue section, but the number of transcripts that can be analyzed in one experiment is limited. Next-generation sequencing approaches have the potential to provide a solution to this problem. However, most sequencing-based approaches require compartmentalization of single cells, which makes it impossible to analyze those cells morphologically, view subcellular attributes, or visualize them in the context of the tissue from which they were obtained. Other sequencing-based platforms rely transferring RNA from a tissue section to a microarray (see, e.g., Bergenstråhle et al. BMC Genomics (2020) 21:482). Such array-based methods have low capture efficiency, relatively low resolution, and a certain amount of spatial information is lost when the RNA molecules diffuse from the tissue to the array. As such, array-based methods are unsatisfactory for a number of applications.

This disclosure provides a way to add spatially-addressable sequence barcodes to nucleic acids, proteins, or other cellular constituents in situ (i.e., within the tissue section). After nucleic acids to which the barcodes have been added are sequenced, the sequences can be mapped to a site in the tissue section using the barcode. This method can be used to provide a transcript and/or protein profile for single cells that are in a tissue section and thus solves for the problems discussed above.

### SUMMARY

Provided herein, among other things, is a method for synthesizing spatially addressed nucleic acid barcodes in or on a cellular sample in situ. In some embodiments the method may comprise: obtaining a cellular sample comprising nucleic acid molecules that are protected by a reversible terminator, deprotecting the nucleic acid molecules in a set of areas of the sample by selectively applying an external stimulus (e.g., light or an electrochemical stimulus) to the set of areas to produce deprotected nucleic acid molecules in the areas, applying a reversible terminator nucleotide to the cellular sample, resulting in addition of a reversible terminator onto the deprotected nucleic acid molecules, optionally removing any unreacted reversible terminator nucleotide from the sample, and repeating the deprotecting, addition and optional removal steps one or more times, thereby producing spatially addressed barcodes in or on the cellular sample. The addition step of the method can be done enzymatically (using a polymerase or terminal transferase) or chemically (using phosphoramidite or H-phosphonate chemistry) in a templated or non-templated manner. In some embodiments, at least some of the repeats the set of areas that are deprotected is different to but overlapping with the prior set of areas that are deprotected.

The method can, for example, be used to add a spatially-addressable barcode to cDNA molecules that are made in situ. In this embodiment, the barcoded cDNA may be sequenced and the cDNA sequences can be mapped to a physical position on the sample by the barcodes associated with those sequences. In addition or as an alternative, the nucleic acid molecules onto which the barcodes are added may be oligonucleotides that are attached to a binding agent such as an antibody or aptamer. In these embodiments, binding agent/oligonucleotide conjugates (i.e., molecules that are composed of a binding agent linked to an oligonucleotide) are bound to sites or epitopes that are in or on cells in the sample, and the oligonucleotide contains a nucleotide sequence (i.e., a binding agent identifier) that identifies the binding agent to which it is attached. In these embodiments, the conjugates may be bound to the sample, barcodes may be added to the oligonucleotides in situ using the method described herein, the barcoded oligonucleotides may be sequenced and the binding sites for the binding agents can be mapped to a physical position on the sample by the barcodes associated with oligonucleotides. In some embodiments, the method may be used to generate RNA profiles and protein profiles for the same cells, thereby providing “multi-omics” data and providing a way to merge images, where the protein binding sites allow for fiduciary alignment and identification of cell types.

### BRIEF DESCRIPTION OF THE FIGURES

The skilled artisan will understand that the drawings described below are for illustration purposes only. The drawings are not intended to limit the scope of the present teachings in any way.

FIG. 1 illustrates some of the principles of an embodiment of the present method.

FIG. 2A illustrates how spatially-addressed barcodes can be built by performing N cycles of deprotection and incorporation.

FIG. 2B illustrates how a sample can be divided into four areas, and four different barcodes can be produced in the different areas.

FIG. 3 schematically illustrates the concept of offset areas.

FIG. 4 illustrates how barcodes can be synthesized on the 5′ end of cDNA that has been made in situ.

FIG. 5 illustrates different cells in a sample can be associated with different barcodes.

FIG. 6 illustrates how molecules from a tissue sample can be embedded in a polymer.

FIG. 7 illustrates a strategy for barcoding that is started by transferring oligonucleotides into a sample from a low resolution array.

FIG. 8 illustrates a strategy for synthesizing a barcode in nuclei in situ using a universal template (a 5 nitroindole base).

FIG. 9 shows a gel (top) and trace (bottom) corresponding to example 1 below.

### DEFINITIONS

Unless defined otherwise herein, all technical and scientific terms used in this specification have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of ordinary skill in the art with the general meaning of many of the terms used herein. Still, certain terms are defined below for the sake of clarity and ease of reference.

As used herein, the term “spatially addressed” and “spatially addressable” refer to sequences that can be mapped to a site or position on a sample, e.g., by x-y-z coordinates.

As used herein, the term “nucleic acid barcode” refers to a sequence of nucleotides that is appended onto one or more target nucleotides. A nucleic acid barcode can be at least 4 nucleotides, e.g., 4-20 nucleotides, in length.

As used herein, the term “spatially addressed nucleic acid barcode” refers to sequences of nucleotides that are appended onto one or more target polynucleotides, where the sequence appended onto each target polynucleotide indicates a position on the sample, e.g., by x-y-z coordinates. A sample that contains spatially addressed nucleic acid barcodes can be subdivided into multiple areas, where each area is associated with a different barcode sequence.

As used herein, the term “cellular sample” is intended to include samples are made by, e.g., growing cells on a planar surface, samples that are made by depositing cells on a planar surface, e.g., by centrifugation, and samples that are made by cutting a three-dimensional object that contains cells into sections and mounting the sections onto a planar surface, i.e., producing a tissue section. The surface upon which a sample may be mounted may be, e.g., glass, metal, ceramic, plastic, etc. If the sample is fixed, it may be fixed using any number of reagents including formalin, methanol, paraformaldehyde, methanol:acetic acid, glutaraldehyde, bifunctional crosslinkers such as bis(succinimidyl)suberate, bis(succinimidyl)polyethyleneglycol etc. A section (e.g., a cryosection) of a tissue sample (e.g., of a fresh frozen tissue sample) that has a thickness in the range of 1-50 um (e.g., in the range of 1-5 um or 5-20 um) is an example of a cellular sample, although there are many alternatives. In some embodiments the cells in the sample may be fixed and/or permeabilized, e.g., using a detergent or a solvent.

As used herein, the term “tissue section” refers to a piece of tissue that has been obtained from a subject and mounted on a planar surface, e.g., a microscope slide.

As used herein, the term “formalin-fixed paraffin embedded (FFPE) tissue section” refers to a piece of tissue, e.g., a biopsy sample that has been obtained from a subject, fixed in formaldehyde (e.g., 3%-5% formaldehyde in phosphate buffered saline) or Bouin solution, embedded in wax, cut into thin sections, and then mounted on a microscope slide.

As used herein, the term “reversible terminator” is a nucleotide at the end of a nucleic acid molecule that contains a group that prevents extension of the nucleic acid molecule at that end. A reversible terminator may be on the 3′ end or the 5′ end of the nucleic acid molecule. The group may be cleaved by an external stimulus, e.g., uv light or an electrochemical or pH change, which makes the terminator “reversible”. The term “reversible terminator” is intended to refer to the types of 3′ reversible terminators on the 3′ end that are used in sequencing-by-synthesis methods (at least some of which are described in Chen at al Genomics, Proteomics & Bioinformatics 2013 11: 34-40) except that reversible terminators used in the current method do not need to be fluorescent. This term is also intended to include the reversible terminators that are used in chemical synthesis methods (e.g., phosphoramidite or H-phosphonate-based oligonucleotide synthesis methods), which are typically called “protecting groups” and are on the 5′ end (see, e.g., Pease et al Proc. Natl. Acad. Sci. 1994 91: 5022 5026 and Egeland et al Nucleic Acids Res. 2005 33: e125).

As used herein, the term “deprotecting” refers to the removal of the blocking group from a reversible terminator. Deprotection allows the nucleic acid on which the reversible terminator is present to be extended. If the reversible terminator is on the 3′ end, deprotecting will typically result in a 3′ hydroxyl, thereby allowing the unblocked nucleic acid to be extended by an enzyme such as a polymerase or terminal transferase. If the reversible terminator is on the 5′ end, deprotecting (or “de-blocking” as it may be called in oligonucleotide synthesis methods) will typically result in a 5′ terminal hydroxyl, that can be reacted with nucleoside phosphoramidite or nucleoside H-phosphonate, thereby allowing the unblocked nucleic acid to be extended via chemical addition.

As used herein, the term “reversible terminator nucleotide” refers to 3′-O-blocked, 3′-unblocked and other reversible terminator deoxynucleotides that are reversibly blocked at the 3′ end (see, e.g., Chen at al Genomics, Proteomics & Bioinformatics 2013 11: 34-40). These reversible terminators typically have a 5′ triphosphate and a cleavable blocking group that prevents addition onto the 3′ end. The term “reversible terminator deoxynucleotide” also refers to nucleoside phosphoramidite and nucleoside H-phosphonates used in synthetic oligonucleotide synthesis methods.

As used herein, the term “removing” refers to any action that results of the elimination of a compound. Removing may include degrading, inactivating or washing away, or any combination thereof.

As used herein, the term “5′ tail”, in the context of a tailed oligonucleotide, refers to a 5′ part of an oligonucleotide that is not complementary to a target and does not hybridize to the target that the 3′ hybridizes to. A tail can be as long as needed, e.g., in the range of 20-100 bases, as desired.

As used herein, the term “oligonucleotide” refers to a multimer of at least 2 nucleotides, e.g., at least 5, at least 10, at least 15 or at least 30 nucleotides. In some embodiments, an oligonucleotide may be in the range of 15-200 nucleotides in length, or more. Any oligonucleotide used herein may be composed of G, A, T and C, or bases that are capable of base pairing reliably with a complementary nucleotide. In some embodiments, an oligonucleotide may additionally contain one or more “universal” bases that can base pair with any of G, A, T and C. Universal bases include 2′-deoxyinosine 2′-deoxynebularine, 3-nitropyrrole 2′-deoxynucleoside and 5-nitroindole 2′-deoxynucleoside, although others are known. Oligonucleotides may be synthetic or may be made enzymatically, and, in some embodiments, are 30 to 150 nucleotides in length. An oligonucleotide may be 10 to 20, 21 to 30, 31 to 40, 41 to 50, 51 to 60, 61 to 70, 71 to 80, 80 to 100, 100 to 150 or 150 to 200 nucleotides in length, for example.

The term “hybridization” or “hybridizes” refers to a process in which a nucleic acid strand anneals to and forms a stable duplex, either a homoduplex or a heteroduplex, under normal hybridization conditions with a second complementary nucleic acid strand and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions includes hybridization at about 42° C. in 50% formamide, 5×SSC, 5×Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “sequencing”, as used herein, refers to a method by which the identity of at least 2 consecutive nucleotides (e.g., the identity of at least 5, at least 10, at least 50 or at least 100 or more consecutive nucleotides) of a polynucleotide are obtained.

The term “next-generation sequencing” refers to the so-called parallelized sequencing-by-synthesis or sequencing-by-ligation platforms currently employed by, e.g., Illumina, Life Technologies, BGI Genomics (Complete Genomics technology), PacBio, Oxford Nanopore, and Roche etc.

The term “duplex,” or “duplexed,” as used herein, describes two complementary polynucleotides that are base-paired, i.e., hybridized together.

The terms “determining,” “measuring,” “evaluating,” “assessing,” “assaying,” and “analyzing” are used interchangeably herein to refer to forms of measurement, and include determining if an element is present or not. These terms include both quantitative and/or qualitative determinations. Assessing may be relative or absolute.

The term “ligating”, as used herein, refers to the enzymatically catalyzed joining of the terminal nucleotide at the 5′ end of a first DNA molecule to the terminal nucleotide at the 3′ end of a second DNA molecule.

The terms “plurality”, “set” and “population” are used interchangeably to refer to something that contains at least 2 members. In certain cases, a plurality may have at least 10, at least 100, at least 100, at least 10,000, or at least 100,000 members.

A “primer binding site” refers to a site to which an oligonucleotide hybridizes in a target polynucleotide or fragment. If an oligonucleotide “provides” a binding site for a primer, then the primer may hybridize to that oligonucleotide or its complement. The term “strand” as used herein refers to a nucleic acid made up of nucleotides covalently linked together by covalent bonds, e.g., phosphodiester bonds.

The term “extending”, as used herein, refers to the extension of a nucleic acid by the addition of nucleotides using a polymerase or by a chemical reaction.

As used herein, the term “splint” refers to an oligonucleotide that hybridize to the ends of two other polynucleotides.

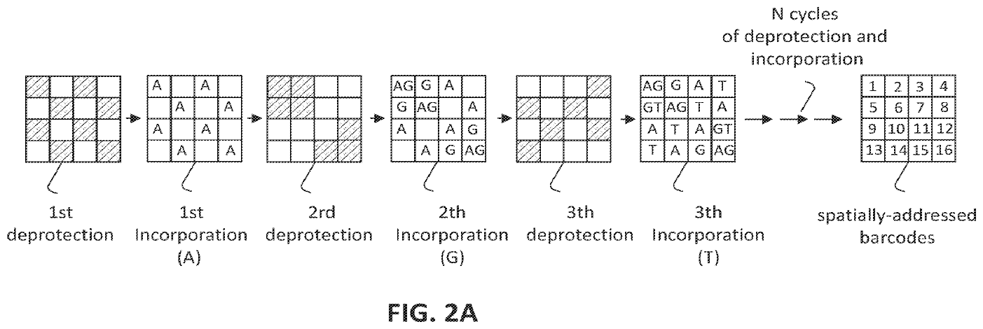

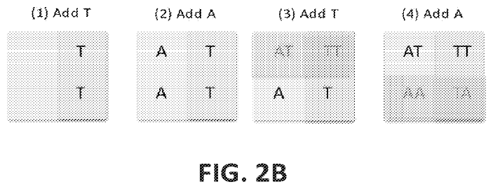

As used herein, the term “overlapping” refers to areas that are off-set to one another, as illustrated in FIG. 2.

The term ‘UMI’ as used herein refers to a unique molecular identifier that makes the individual molecules in a library unique, or the library unique.

Other definitions of terms may appear throughout the specification.

### DETAILED DESCRIPTION

Provided herein, among other things, is a method for synthesizing spatially addressed nucleic acid barcodes in or on a cellular sample in situ. In some embodiments, the method may be performed on a cellular sample (e.g., a tissue section, synthetic tissue, printed cells, organoids etc.) comprising nucleic acid molecules that are protected by a reversible terminator. As will be explained in greater detail below, the sample may be made in a variety of different ways, e.g., by hybridizing, ligating, or binding one or more oligonucleotides that are protected by a reversibly terminator to the sample or reversibly terminating nucleic acid molecules that are native to the sample. Depending on how the method is implemented (e.g., depending on whether the reversible terminator is on the end of a nucleic acid (e.g., an oligonucleotide) that is added to or made in the sample and whether the reversibly terminator is at the 5′ or 3′ of the nucleic acids, etc.) the 3′ hydroxyls that are present in nucleic acids that are endogenous to the sample may be blocked first, e.g., by irreversibly capping them with a dideoxy terminator or the like. The barcode may be directly synthesized onto DNA, RNA, cDNA, and synthetic DNA, e.g., oligonucleotides, as desired. The reversible terminator does not need to be labeled in this method because the method is not an in situ sequencing method. As such, in some embodiments, the reversible terminator may be unlabeled (i.e., not fluorescent). In other embodiments, the reversible terminator may be labeled (i.e., may contain a fluorophore).

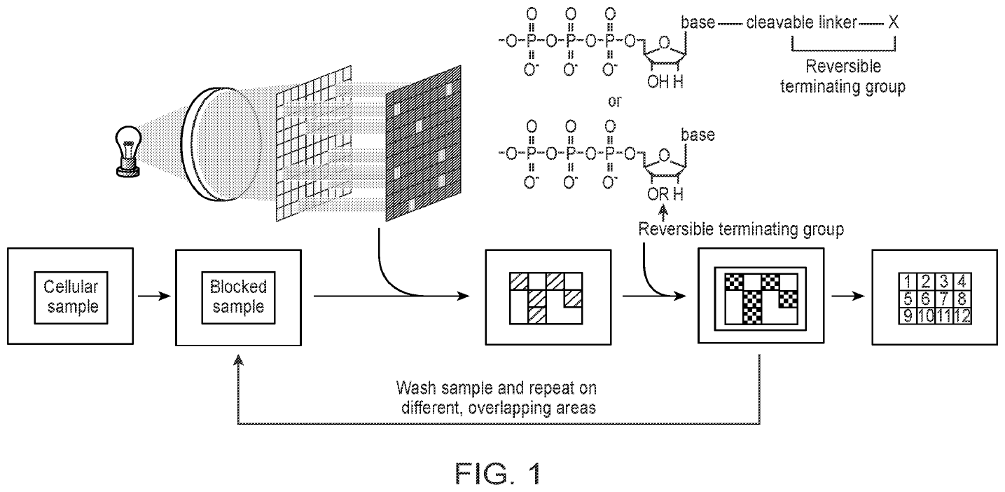

FIGS. 1-3 illustrate some principles of the method. As illustrated in FIG. 1, the method may comprise deprotecting the nucleic acid molecules in a set of areas of the sample (not the entire sample, but rather selected areas within the sample to which the different barcodes will be added) by selectively applying an external stimulus to the set of areas to produce deprotected nucleic acid molecules in the areas. As illustrated, this step may be implemented by light using a mask. However, as will be described in greater detail below, other methods (e.g., maskless or projection methods) may be used. The set of areas that are deprotected in this step of the method may comprise at least 10, (e.g., at least 100, at least 500, at least 1000, at least 5,000, at least 10,000, at least 50,000, at least 100,000, at least 500,000, or at least 1M) areas, at least some of which will not be contiguous with another area. In many cases, the areas are the same may be the same size and shape, e.g., square or rectangular. In some embodiments, only select regions or regions of interest (ROI) of the sample are barcoded. Such approach would allow the analysis of specific regions, pre-determined area(s) of the sample, enabling the analysis of select cells. In some embodiments, the selection of the specific area(s) is done by an external stimulus e.g. light. FIG. 2 illustrates how barcodes can be built using multiple rounds of deprotection and incorporation (addition) by deprotecting a different set of areas in each cycle.

In some embodiments, the method may comprise: (a) obtaining a cellular sample comprising nucleic acid molecules; (b) introducing a primer to the sample, (c) protecting an area of the sample, (d) deprotecting the primer in a set of areas of the sample by selectively applying an external stimulus to the set of areas to produce deprotected nucleic acid molecules in the areas, (e) applying a reversible terminator nucleotide to the cellular sample, resulting in addition of a reversible terminator onto the deprotected primer, (f) optionally removing any unreacted reversible terminator nucleotide after step (e); and (g) repeating steps (d)-(f) one or more times, to produce spatially addressed barcodes that are attached to nucleic acid molecules or derivatives that are in or on the cellular sample.

In other embodiments, the method may comprise: (a) obtaining a cellular sample comprising nucleic acid molecules, (b) protecting the nucleic acid molecules with a blocking group, (c) deprotecting the nucleic acid molecules in a set of areas of the sample by selectively applying an external stimulus to the set of areas to produce deprotected nucleic acid molecules in the areas, (d) applying a reversible terminator nucleotide to the cellular sample, resulting in addition of a reversible terminator onto the deprotected nucleic acid molecules, (e) optionally removing any unreacted reversible terminator nucleotide after step (e); and (f) repeating steps (c)-(e) one or more times, to produce spatially addressed barcodes that are attached to nucleic acid molecules that are in or on the cellular sample.

The dimensions, number and density of the areas may vary. The standard size of a tissue sample is 6 mm×6 mm although the present method can be adapted to samples that have a different size. The deprotected areas may vary in size and, in some embodiments, may have a diameter of in the range of 1 nm to 10 mm, e.g., 10 nm to 1 mm, 100 nm to 100 um or 100 nm to 10 um. Likewise, depending on the method used to pattern the deprotection, there may be at least 10, at least 100, at least 1000 or at least 10,000 deprotected areas in each repeat. The density of deprotected areas can range, too, e.g., from 0.1 areas/mm² to 100M areas/mm², e.g., 10² area/mm² to 10⁷ areas/mm² or 10³ area/mm² to 10⁶ areas/mm². The areas may or may not be separated by a gap.

After areas on the sample have been deprotected, a reversible terminator nucleotide is applied to the cellular sample. In this step, the reversible terminator nucleotide is applied to a region that contains the areas that have been deprotected as well as areas that are in-between those areas (which have not been deprotected). This may be done by flooding the sample with the reversible terminator nucleotide and any other necessary reagents for adding the reversible terminator to deprotected nucleic acid molecules (e.g., an enzyme such as a polymerase or terminal transfers, as well as any other necessary reagents and cofactors, etc.). In this step, the reversible terminator nucleotide will only react with nucleic acid molecules that have been deprotected in the prior step (and not with protected nucleic acid molecules that are in other areas or capped molecules in the same area as the deprotected nucleic acids). As such, this step of the method results in the addition of a reversible terminator (i.e., part of the reversible terminator nucleotide) onto the deprotected nucleic acid molecules. As would be apparent, this addition reaction does not occur in areas that have not been deprotected, or capped molecules within the deprotected areas (which may be endogenous to the sample).

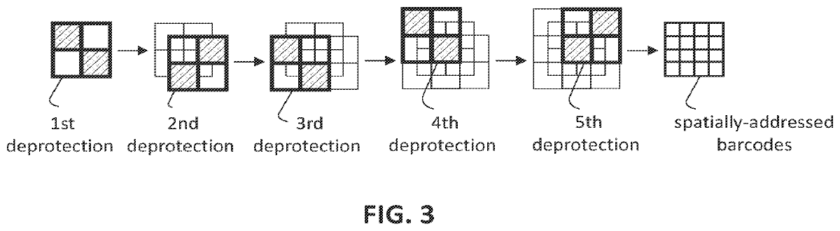

After optionally removing any unreacted reversible terminator nucleotide (i.e., any reversible terminator nucleotide that has not been added to the end of a nucleic acid), by, e.g., washing the unreacted reversible terminator nucleotide from the sample, degrading unreacted reversible terminator nucleotide or inactivating the unreacted reversible terminator nucleotide, the deprotection, addition and optional washing steps may be repeated one or more times (e.g., at least twice, at least 3 times, at least 5 times, at least 10 times or at least 20 times) to produce spatially addressed barcodes that are attached to nucleic acid molecules that are in or on the cellular sample. In this step, in some embodiments at least some (e.g., at least 2, at least 5 or at least 10) of the repeats the areas that are deprotected may be different to but overlapping with the prior set of areas that are deprotected. This concept is illustrated in FIG. 3 and may result in a 1-50% overlap between the areas that are deprotected in consecutive repeats. Illustrated by example, if 1000 areas are deprotected in one repeat and 1000 areas are deprotected in the next repeat, then there may be an overlap between 50-500 of the areas. In this example, at the end of the two repeats, two nucleotides would be added to areas that are deprotected in consecutive repeats (i.e., in an area of overlap) but only one nucleotide would be added to the areas that are deprotected in a single repeat (in non-overlapping areas). As would be apparent, different nucleotides (i.e., G, A, T, or C, or an analog thereof) may be added in the different repeats. For example, if a single type of reversible terminator (corresponding to G, for example) is added in the initial addition step (i.e., in step c), then the first repeat may be done with another type of reversible terminator (corresponding to A, T, or C) and likewise in consecutive repeats. However, in some cases, the same reversible terminator (corresponding to G, for example) may be added in consecutive steps. In other embodiments, the areas that are deprotected are the same in each cycle (i.e., they are not off-set).

As illustrate in FIG. 1, the method results in a section that is divided into multiple areas (e.g., areas 1-12), wherein the nucleic acids in the different areas have different barcodes, i.e., the nucleic acids within an area have a barcode that is distinguishable from the barcodes from other areas. For example, the barcode added to the nucleic acids in area 1 has a sequence that is distinguishable from the sequences of the barcodes in areas 2-12, and so on. As such, sequences of the barcoded nucleic acids from areas 2-12 can be mapped to a particular area on the sample by the barcode.

FIG. 3 illustrates the principle of “offsetting” areas. Off-setting the areas that are deprotected in consecutive deprotection cycles (i.e., by off-setting the mask, digital microarray mirror, projector or patterned electrodes) creates an offset between some of the areas that are deprotected in one cycle relative to the next. Off-setting the areas that are deprotected allows barcodes to be made at a higher density than the resolution of the deprotection system used. By way of example, if the deprotection system used in the method (i.e., the mask, digital microarray mirror, projector or patterned electrodes) has a resolution of 10 uM and deprotects areas that are 10 uM×10 uM in size, then overlapping the areas that are deprotected in consecutive cycles can result in at least 2, at least 4, at least 8, or at least 16 different barcodes in each area, which is well beyond the resolution of the system. For example, if the x and y off-sets are half the dimension of the areas that are initially deprotected, then the resolution of the barcodes can be doubled. This principle can be used to increase the resolution of barcode synthesis by at least 2×, at least 4×, at least 8×, at least 16×, at least 32×, at least 64×, at least 128× or at least 256× relative to the resolution of deprotection system. For example, in some embodiments, the offset in each repeat can be less than 0.5× the dimension of the areas that are deprotected in the initial deprotection step, thereby increasing the number different barcodes by at least 4×. In some embodiments, the offset in each repeat can be in the range of 0.01-0.5× (e.g., 0.02 to 0.2×) the size of the areas that are deprotected in the initial deprotection step, leading to 4 to 10,000 (e.g., 25 to 2,500) barcodes in each area. As such, in some embodiments, the selective application of the external stimulus in the initial deprotection step may be done using a deprotection system (the mask, digital microarray mirror, projector or patterned electrodes), and the deprotection system used in one or more of the repeats may be off-set relative to the initial deprotection step. In these embodiments, the areas that are deprotected in the initial deprotection step or any of the repeats are different to but overlap with the prior set of areas that are deprotected. In some embodiments, the areas that are deprotected do not overlap with previous deprotection steps. For example, specific regions (set A) are deprotected, a specific nucleotide is incorporated in the set A regions. Subsequently, a new set of specific regions (set B) are deprotected, a specific nucleotide is incorporated different from the nucleotide in set A. The process can be repeated as many times as desired to uniquely barcode specific regions of the cellular sample. In other embodiments, the areas that are deprotected do overlap with previous deprotection steps.

In some embodiments, the method may be additionally used to add a UMI (i.e., a unique sequence) onto every nucleic acid molecule, which would require multiple rounds of addition that each use multiple types of reversible terminator (e.g., a mixture of G, A, T and C). Such UMIs may be used to identify duplicate sequence reads (i.e., identical sequence reads from the same original molecule) and, as such, find use in applications in which nucleic acids are quantified.

FIGS. 2A and 2B illustrate this principle of how a barcode can be built using this method. FIG. 2A shows how spatially-addressed barcodes can be built by performing N cycles of deprotection and incorporation. As shown, in some embodiments the areas that are deprotected may overlap with previous deprotection steps. In other embodiments the areas that are deprotected may be the same as the previous deprotection steps. In the example shown in FIG. 2B, four different barcodes are produced in four areas using two nucleotides. In practice, the barcodes may be longer (e.g., in the range of 4-25 nucleotides) and more nucleotides (typically all four) may be used. In some embodiments, two or more nucleotides of the reversible terminator nucleotides (e.g., A and T, or G and C, etc.) could be added in the initial addition step or one or more of the repeats, thereby resulting in a degenerate barcode in one or more of the regions. In some embodiments, the reversible terminator nucleotide added in one or more of the repeats is different to the reversible terminator nucleotide in a prior repeat. In some embodiments, a single type of reversible terminator (e.g., a nucleotide corresponding to G, A, T or C) is added in the initial addition step and a single type of reversible terminator (e.g., a nucleotide corresponding to G, A, T or C) is added in all of the repeats. In some embodiments, part of the barcode synthesized some or all of the areas may be the same, thereby adding a common sequence onto some or all of the nucleic acids. In these embodiments, that part of the barcode may be an adapter sequence and may be used to amplify all of the barcoded nucleic acids by PCR using a single primer pair (or nested primer). Alternatively, that part of the barcode may be used as a sample identifier.

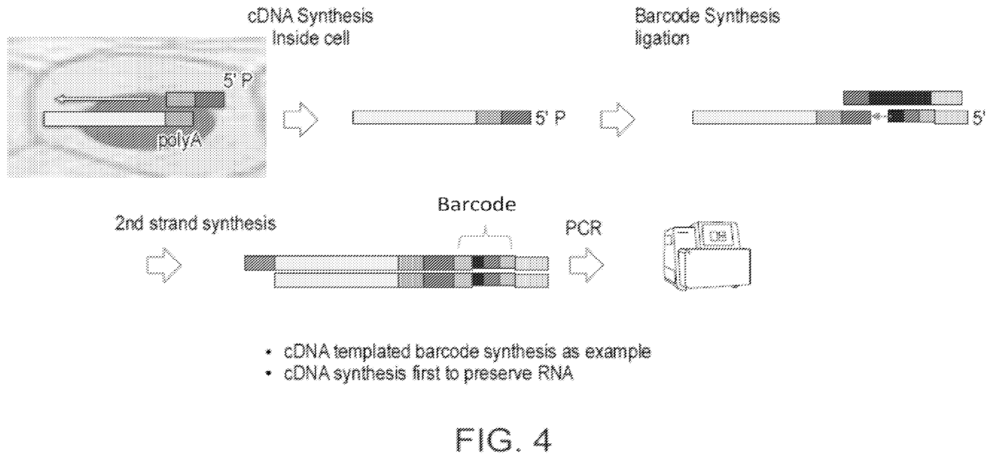

The cellular sample comprising nucleic acid molecules that are protected by a reversible terminator may be made in a variety of different ways. In some embodiments, the sample may be made by hybridizing an oligonucleotide that is protected by a reversibly terminator to a nucleic acid that is endogenous to the sample or a copy of the same (e.g., cDNA) that is produced in situ. In these embodiments, the reversibly terminated oligonucleotide may hybridize directly to an endogenous nucleic acid or copy of the same, or indirectly to an endogenous nucleic acid or copy of the same via a splint. An example of the latter is shown in FIG. 4. Alternatively, an oligonucleotide that is protected by a reversible terminator could be ligated to the nucleic acid that is endogenous to the sample or a copy of the same (e.g., cDNA) that is produced in situ. In these embodiments, the reversibly terminated oligonucleotide may be directly ligated to the endogenous nucleic acid or copy of the same. In addition, an oligonucleotide that is protected by a reversible terminator could be bound to the sample via a binding moiety, e.g., an antibody, aptamer or oligonucleotide probe. Oligonucleotide may be linked to binding agents using any convenient method (see, e.g., Gong et al., Bioconjugate Chem. 2016 27: 217-225 and Kazane et al. Proc Natl Acad Sci 2012 109: 3731-3736). Such conjugates (without the reversible terminator) have been used for the multiplexed analysis of cellular samples (see Samusik et al Cell 2018 174: 968-981.e15) and can be readily adapted herein. In this latter embodiment, the oligonucleotides may be linked to protein binding agents and, as such, the present method may be used to analyze protein epitopes that are on or in a cell. Alternatively, the cellular sample could be treated by reversibly terminating all of the nucleic acid molecules that are native to the sample or copies of the same, e.g., by adding a reversible terminator to the 3′ end of nucleic acids in the sample using, for example, a terminal transferase or polyA or polyG polymerase.

In some embodiments, particularly if addition is to the 3′ end, the method may comprise (i) enzymatically blocking the 3′ hydroxyls that are present in nucleic acids that are endogenous to the sample (using, e.g., a dideoxy terminator), and (ii) binding an oligonucleotide that is protected at the 3′ end by the reversible terminator to the sample. In these embodiments, (i) and (ii) may done in either order.

In some embodiments, the addition step may be non-templated (meaning that there is no underlying nucleic acid “template”) for the addition. These embodiments may be catalyzed by a terminal transferase (for the 3′ end) or via chemical addition (for the 5′ end). Examples include, but not limited, de novo DNA synthesis using polymerase nucleotide conjugates (see, e.g., Palluk et al Nature Biotechnology 2018 36: 645-650).

In alternative embodiments, the addition step may be templated (meaning that there may be an underlying nucleic acid “template” that may contain one or more universal nucleotides that can base pair with any of G, A, T and C, e.g., 2′-deoxyinosine 2′-deoxynebularine, 3-nitropyrrole 2′-deoxynucleoside and 5-nitroindole 2′-deoxynucleoside or the like. These embodiments may be implemented by a polymerase e,g., an engineered polymerase (e.g. Ichida et al Nucl. Acids Res. 2005 33:5219-25. Templated methods can have advantages because in some cases another oligonucleotide can be hybridized to the template downstream of the universal bases and, after the barcode has been added the barcode can be ligated to the downstream oligonucleotide. In some embodiments, the template can contain random or semi random base pairs to accept any incoming nucleotide or terminating base. In some embodiments, the template contains a unique sequence that uniquely binds to the analyte or derivative of the analyte. In some embodiments, the template can contain universal bases or universal bases alternating with other bases. Examples include, but not limited to, cyclic reversible termination (see, e.g., Hoff et al ACS Synth. Biol. 2020, 9, 2, 283-293 and Hoff et al bioRxiv 2019 561092).

FIG. 4 shows an embodiment of how a barcode can be added to cDNA in a templated addition reaction. As illustrated in FIG. 4, this embodiment of the method may comprise hybridizing a tailed reverse transcription primer (i.e., an oligonucleotide that has a 3′ end composed of oligo(dT), a random sequence or gene-specific sequence, and a 5′ end that does not hybridize to the RNA) to RNA in the cellular sample in situ. The reverse transcription primer may be extended in situ (in a reaction that contains NTPs and reverse transcriptase) to produce cDNA products that comprise the sequence of the tailed reverse transcription primer (at the 5′ end) and first strand cDNA. After first stand cDNA synthesis, a splint oligonucleotide and a splint primer are hybridized to the cDNA products in situ, where the splint oligonucleotides comprise internal universal nucleotides and the primer has a 3′ reversible terminator. In these embodiments, the cDNA products, the splint oligonucleotide and the primer hybridize to one another to produce a complex that contains a gap between the 5′ end of the cDNA and the 3′ end of the primer, wherein the gap is across from the universal nucleotides. As would be recognized, the splint primer could be pre-hybridized to the splint oligonucleotide prior to hybridizing the splint oligonucleotide to the cDNA in this embodiment. Indeed, in this embodiment the reverse transcription primer, the splint oligonucleotide and the splint primer could be pre-hybridized to one another prior to cDNA synthesis. The reverse transcription primer can include an UMI, barcode, common sequence, amplification primer, sample index etc. In some embodiments, different cellular samples are prepared individually with a unique sample index or barcode. The barcoded libraries of the different cellular samples are pooled, and sequencing of the libraries for the samples is performed simultaneously. In either event, the barcode may be added to the 5′ end of the cDNA using the present method, i.e., by adding nucleotides to the 3′ end of the splint primer across from the universal nucleotides to make an extension product and (ii) sealing the extension product to the 5′ end of the extension products by ligation. As such, this embodiment of the method may make use of a polymerase and a primer to fill in and then seal the splint primer to the cDNA in a gap fill reaction (see, e.g., Mignardi et al, Nucleic Acids Res. 2015 43: e151). In some embodiments, the primer is non-natural or synthetically made. In some embodiments, the primer is used for barcode synthesis. In some embodiments, the analyte or derivative, or nucleic acids, act as a primer.

In embodiments in which the addition is done enzymatically, i.e., using a terminal transferase or a polymerase, the reversible terminator nucleotide applied to the sample may comprise a group that can be cleaved by the external stimulus to produce a 3′ hydroxyl. For example, the reversible terminator may comprise a number of different groups, including but not limited to, a 2-nitrobenzyl group, a 2-nitrobenzyl-modified thymidine analog group, a 2-nitrophenyl group, a phenacyl group, a 6-nitropiperonyl group, a 9-anthrylmethyl group, a 3′-O-(2-nitrobenzyl) group, a 3′-O-(4,5-dimethoxy-2-nitrobenzyl) group, etc., any of which can be cleaved by light (e.g., light at a wavelength of 100-400 nm) to either allow access to the 3′ hydroxyl or to produce a 3′ OH. In some embodiments, two or more terminators can be de-blocked with two or more different wavelengths, one specific wavelength per terminator. This deprotection chemistry may be adapted from any of a variety of sequencing-by-synthesis technologies. Deprotection chemistries that could be employed herein include those described in Klan et al (Chem. Rev. 2013 113: 119-191), Mathews et al (Org. Biomol. Chem. 2016 14: 8278-8288), Bochacova et al (Org. Biomol. Chem. 2018 16: 1527), Gardner et al (Nucleic Acids Research 2012 40: 7404-7415) and Wu et al (Proc. Natl Acad. Sci. 2007 104: 16462-16467). As would be understood, the nucleotides used in the present method do not need to be fluorescent and, as such, the fluorescent labels can be eliminated from any reversible terminator nucleotide that may be used in a sequencing-by-synthesis method. See also WO2020120442.

Examples of 3′-O-blocked reversible terminator nucleotides are described below.

In some embodiments, the reversible terminator nucleotide may comprise a 3′-O-(2-nitrobenzyl) group attached to the 3′ O of the sugar moiety of the nucleotide, which can be cleaved off to produce a 3′ hydroxyl by light at a wavelength of 100-400 nm. This reaction chemistry is described in Wu et al supra and Mathews et al supra and can be adapted for use herein.

In another example, the reversible terminator nucleotide may comprise a 3′-O-(4,5-dimethoxy-2-nitrobenzyl) group attached to the 3′ 0 of the sugar moiety of the nucleotide, which can be cleaved off to produce a 3′ hydroxyl by light at a wavelength of 100-400 nm. This reaction chemistry is described in Mathews et al supra and can be adapted for use herein.

Examples of 3′ unblocked reversible terminator nucleotides are described below.

In some embodiments, the reversible terminator nucleotide may comprise a 2-nitrobenzyl group attached to the base, which can be cleaved off by light at a wavelength of 100-400 nm to unblock the nucleotide. This reaction chemistry is described in Gardner et al supra and Klan et al supra and can be adapted for use herein.

In another example, the reversible terminator nucleotide may comprise a 2-nitrophenyl group attached to the base, which can be cleaved off by light at a wavelength of 100-400 nm to unblock the nucleotide. This reaction chemistry is described in Gardner et al supra and can be adapted for use herein.

In another example, the reversible terminator nucleotide may comprise a 6-nitropiperonyl group attached to the base, which can be cleaved off by light at a wavelength of 100-400 nm to unblock the nucleotide. This reaction chemistry is described in Bochacova et al supra and can be adapted for use herein.

In another example, the reversible terminator nucleotide may comprise a 6-nitropiperonyl group attached to the base, which can be cleaved off by light at a wavelength of 100-400 nm to unblock the nucleotide. This reaction chemistry is described in Bochacova et al supra and can be adapted for use herein.

In another example, the reversible terminator nucleotide may comprise a 9-anthrylmethyl group attached to the base, which can be cleaved off by light at a wavelength of 100-400 nm to unblock the nucleotide. This reaction chemistry is described in Bochacova et al supra and can be adapted for use herein.

In some embodiments, a tert butyl group may reside between the nucleotide base and the above mentioned groups in order to increase the efficiency of photocleavage. For example, the reversible terminator nucleotide may comprise a 2-nitrobenzyl group followed by a tert-butyl group attached to the base, which can be cleaved off by light at a wavelength of 100-400 nm to unblock the nucleotide. This reaction chemistry is described in Gardner et al supra and can be adapted for use herein.

As noted above, in some embodiments, the reversible terminator may be added to the 5′ end of the nucleic acid molecules. In these embodiments, the nucleic acid molecules of the sample may be protected by a reversible terminator at the 5′ end and the addition of step adds to the 5′ end of the deprotected nucleic acid molecules. In these embodiments, the cellular sample may be made by binding oligonucleotides that are protected at the 5′ end by a reversible terminator to the sample, e.g., via hybridization, ligation or via binding agents to which the oligonucleotides are tethered. In these embodiments, the addition may be done using phosphoramidite or H-phosphonate addition chemistry. This reaction chemistry is commonly employed in oligonucleotide synthesis, and can be adapted from a variety of publications, including Pease et al (Proc. Natl. Acad. Sci. 1994 91: 5022 5026) and Egeland et al (Nucleic Acids Res. 2005 33: e125). In these embodiments, the protecting group on the nucleotide may include 5′-O-DMT, nitroveratryloxycarbonyl, NVOC), 4,4′-dimethoxytrityl (DMT) or 5′-O-(α-methyl-6-nitropiperonyloxycarbonyl) (MeNPOC). In some embodiments, the primer is blocked or protected and can be de-blocked or deprotected to enable elongation.

The external stimulus that is applied to the sample in order to deprotect the protected nucleic acid molecules can be light stimulus e.g., uv light that may be at a wavelength of 360-480 nm, an electrochemical stimulus (see, e.g., Egeland et al Nucleic Acids Res. 2005 33: e125) or a photoelectrochemical synthesis (see, e.g, Chow et al Proc. Natl. Acad. Sci. 2009 106: 15219-24) or pH change. In order to pattern the external stimulus, the external stimulus may be selectively applied to specific areas by a mask (Lietard et al J. Vis. Exp. 2019 e59936), optical projection (see Agbavwe et al Journal of Nanobiotechnology 2011: 9; Haldar et al, AIP Conference Proceedings 2115, 030219 (2019)), a digital microarray mirror, a laser or laser scanner, optical device, mask or mask less optical system, or patterned electrodes. In any embodiment the reversible terminator of step (a) or at least one of the reversible terminators added in step (c) comprises an affinity tag, e.g., biotin or desthiobiotin, for example, which can be used to isolate product molecules that contain a barcode from other molecules prior to analysis. In some embodiments, the affinity tag can be attached to the oligonucleotide onto which the barcode is added. In some embodiments, the barcoded libraries are extracted using electrophoresis, magnetism, heat, lysis, digestion, etc., prior to further analysis.

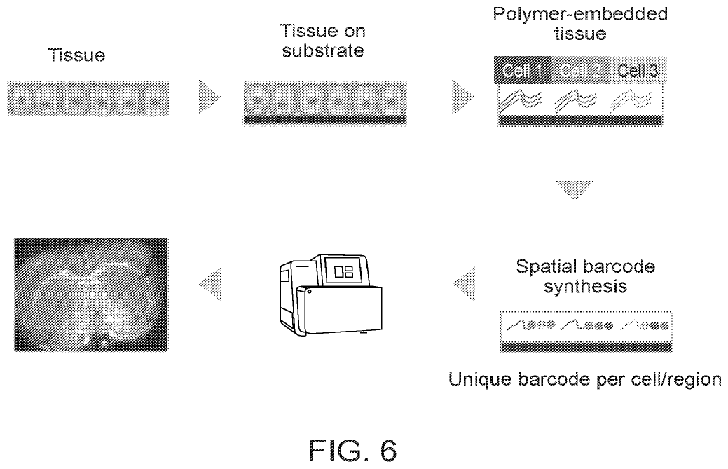

In some embodiments, the sample can be embedded in a polymer. In some configurations only the inside of the cells are polymerized (see Arnaud Chemical and Engineering News 2019 97: 16). In some embodiments, the polymerization is reversible. The tissue is optionally dissociated after embedding and single cell analytes are immobilized spatially at the location of origin. The analytes can be immobilized chemically, non-chemically, through entrapment, covalently or non-covalently to the polymer matrix. After immobilization of the analytes to the polymer matrix, the tissue can be dissociated. One advantage of such approach is that diffusion limitations imposed by the heterogeneous nature of the tissue can be eliminated, enabling uniform and efficient barcode synthesis to each section of the tissue. An example workflow is shown in FIG. 6. In some embodiments, the polymer embedded tissue may be subjected to expansion, stretching, or enlargement before spatial barcode synthesis. In some embodiments, the expansion is only used during barcode synthesis and no spatial information is directly recorded during time of expansion. Methods and compositions of expansion in the context of spatial single cell analysis have been described in the literature (Alon et al., Science 371, 481, and references cited herein).

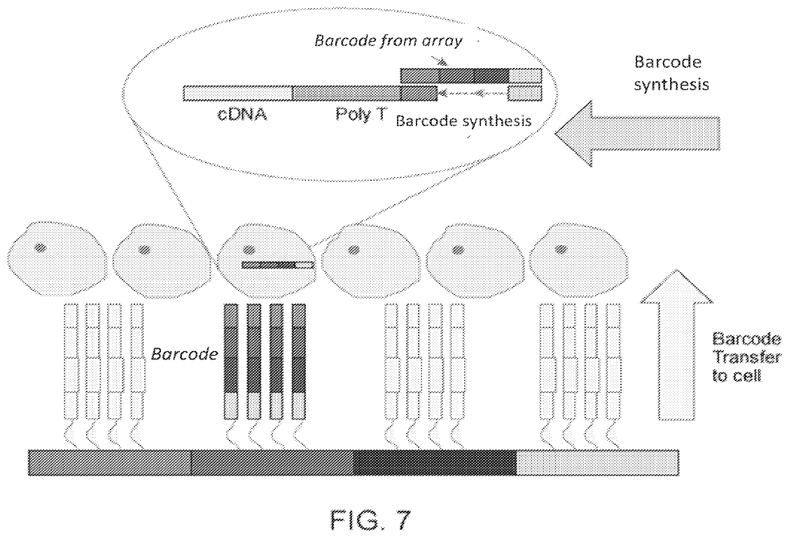

In additional embodiments, the barcoding process could be initiated at a lower resolution using conventional array of barcoded primers, as shown in FIG. 7. In this embodiment, the sample may be placed on the array, the oligonucleotides may be cleaved from the array and transferred into the sample, and higher resolution barcode synthesis can be initiated using the present method, using the transferred oligonucleotides as a template. For example, barcoded oligonucleotides (e.g. barcoded RT primers) from an array may be released and bind to analytes in the tissue or polymer-embedded tissue thereby generating barcoded libraries. These barcoded libraries are further processed and sequenced ex situ. In some embodiments, these orthogonal barcoded methods are combined with barcode synthesis. For example, barcoded oligos from an array are further barcoded using the spatial synthesis methods as described above. In some embodiments, the barcoded primers of the arrays are applied and immobilized throughout the cellular sample and used to capture analytes, analyte derivatives, or nucleic acid molecules of interest. In some embodiments, the barcoded primers are amplified (e.g., rolling circle amplification, PCR, linear amplification etc.) prior to capturing nucleic acid molecules of interest. For example, circular barcoded primers can be amplified using rolling circle amplification. In some embodiments, the barcoded primer is used to initiate amplification of linear or circular nucleic acids.

Similarly, in another embodiment, longer barcodes could be made by ligating short oligonucleotides (e.g., a 2-mer or 3-mer or longer) using the reversible terminator approach described above. These embodiments may involve deprotecting the sample, as described above, and adding a 2-mer or 3-mer to the growing chain (where the 2-mer or 3-mer has a reversibly terminated 3′ end). This ligation may be templated (using a splint template that contains universal bases, as described above) or non-templated, as desired. Alternatively, one could ligate 2-mer or 3-mers or longer using simple overhangs, again deblocking each round.

However, if the extension is performed, the addition may result in a phosphodiester bond between the new nucleotide and the deprotected nucleic acid, thereby allowing the barcode and the nucleic acid to which the barcode has been added to be amplified (by PCR, for example).

In addition to the labeling methods described above, the sample may be stained using a cytological stain, either before or after performing the method described above. In these embodiments, the stain may be, for example, phalloidin, gadodiamide, acridine orange, bismarck brown, barmine, Coomassie blue, bresyl violet, brystal violet, DAPI, hematoxylin, eosin, ethidium bromide, acid fuchsine, haematoxylin, hoechst stains, iodine, malachite green, methyl green, methylene blue, neutral red, Nile blue, Nile red, osmium tetroxide (formal name: osmium tetraoxide), rhodamine, safranin, phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead(II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, vanadyl sulfate, or any derivative thereof. The stain may be specific for any feature of interest, such as a protein or class of proteins, phospholipids, DNA (e.g., dsDNA, ssDNA), RNA, an organelle (e.g., cell membrane, mitochondria, endoplasmic recticulum, golgi body, nuclear envelope, and so forth), or a compartment of the cell (e.g., cytosol, nuclear fraction, and so forth). The stain may enhance contrast or imaging of intracellular or extracellular structures. In some embodiments, the sample may be stained with haematoxylin and eosin (H&E). In these embodiments, the sample may be analyzed by microscopy to produce one or more images of the sample, prior to, during, or after adding barcodes to the sample. In some embodiments, the imaging is used to visualize the morphology of the cellular structures e.g., light microscopy. In some embodiments, the information obtained from light microscopy is used to guide the location, position, and analyze the barcoded nucleic acids onto the cellular sample.

In some embodiments, the cellular sample may be labeled with antibodies and analyzed by microscopy in order to visualize cellular features, e.g., via fluorescence, phase-contrast, white light etc. In some embodiments and as will be described in greater detail below, the antibodies-oligonucleotide conjugates may be used, where the antibodies bind to epitopes in or on the cells and the barcodes are added to the oligonucleotides, These embodiments can be processed in the same workflow as described as above. In some embodiments, RNA and protein epitopes may be analyzed in the same experiment, thereby providing multi-omics data

In some embodiments, the present method may be implemented on a flow cell or microfluidic device, thereby making the addition of reagents and wash automatic or semi-automatic. In some embodiments, the flow cell may be sub-divided into multiple sections, thereby enabling the delivery of two or more different reagents and solutions, terminators, etc. per cycle.

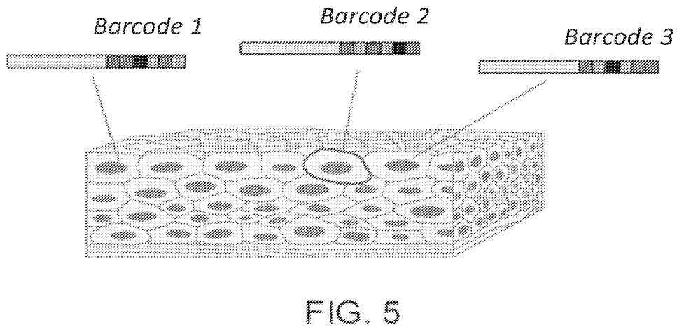

After the nucleic acids in or on the sample have been barcoded using the method described above, the barcoded nucleic acids may be collected, sequenced en masse, and the sequences may be mapped to a position in the sample using the appended barcodes. The barcode serves as an address for the sequence. In some embodiments, the sequences from a particular cell in the sample can be resolved from sequences from other cells in the sample. This concept is illustrated in FIG. 5. In these embodiments, the method may further comprise sequencing the barcodes produced and at least part of the nucleic acid molecules to which they are attached, or an amplification product thereof, and mapping the sequenced nucleic acid molecules to a site in or on the cellular sample using the barcode to which it is attached. The barcoded nucleic acids can have PCR primer binding sites, thereby facilitating amplification of the barcoded nucleic acids. Alternatively, the barcoded nucleic acids may have an affinity tag, thereby facilitating their enrichment. In some embodiments, a subset of the barcoded nucleic acids may be enriched and sequenced, e.g., by enriching for barcoded nucleic acids that have a particular barcode, or nucleic acid sequence.

The barcoded nucleic acids may be sequenced by any suitable system Illumina's reversible terminator method, Roche's pyrosequencing method (454), Life Technologies' sequencing by ligation (the SOLiD platform), Life Technologies' Ion Torrent platform or Pacific Biosciences' fluorescent base-cleavage method and any other platforms e.g. Oxford Nanopore. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) English (PLoS One. 2012 7: e47768) and Morozova (Genomics. 2008 92:255-64), which are incorporated by reference for the general descriptions of the methods and the particular steps of the methods, including all starting products, reagents, and final products for each of the steps.

The sequencing step may be done using any convenient next generation sequencing method and may result in at least 10,000, at least 100,000, at least 500,000, at least 1 M at least 10 M at least 100 M, at least 1 B or at least 10 B sequence reads per reaction. In some cases, the reads may be paired-end reads.

The method may be used to map endogenous nucleic acid molecules. For example, the method may be used to map mRNA sequences. In this example, which is illustrated in FIG. 4, cDNA is synthesized in situ using a tailed primer, and spatially addressable barcodes are added to the 5′ ends of the cDNA molecules using a splint template using the method described above. In this example, first strand cDNA may be collected, second strand cDNA may be produced, and the second strands amplified by PCR and then sequenced. The barcodes appended to the cDNA allow the cDNA sequences to be mapped to a site on the sample.

In addition, the method may be used to map endogenous epitopes, which may be extracellular or intracellular. In these embodiments, a binding agent, e.g., an antibody or aptamer, that is non-covalently (e.g., via a streptavidin/biotin interaction) or covalently (e.g., via a “click” reaction (see, e.g., Evans Aus. J. Chem. 2007 60: 384-395) or the like) linked to a single-stranded reversibly terminated oligonucleotide in a way that the binding agent can still bind to its binding site is used to label the sample. This step may involve contacting the sample (e.g., an FFPE section mounted on a planar surface such as a microscope slide) with all of the binding agents, en masse under conditions by which the binding agents bind to complementary sites (e.g., protein epitopes) in the sample. In some embodiments, the binding agents may be cross-linked to the sample, thereby preventing the binding agents from disassociating during subsequent steps. After the sample has been bound to the binding agents, the barcodes can be synthesized on the oligonucleotides or derivative. The barcoded oligonucleotides can then be released or amplified and then sequenced. In some cases, the oligonucleotides may also contain an identifier sequence that identifies the antibody to which it is bound. In these embodiments, the method can be performed using at least 10, at least 50 or at least 100 different antibodies (i.e., antibodies that recognize epitopes on different proteins). The sequences should contain the added barcode as well as the identifier sequences, thereby allowing the binding site for each antibody to mapped. These embodiments could be used to resolving cellular components and structures. Combining a cellular component barcode (barcode specific to a specific cell component) with spatial barcode synthesis provides a platform for spatially resolving components and cellular sub-structures. Intercalating barcodes or oligos or antibody-oligo conjugates against cellular structure(s) can be used to spatially label and barcode components, including, but not limited, organelles, membranes, nuclei, Golgi apparatus, lysosome, peroxisome, pores, ER, centrioles, mitochondria, ribosomes etc. This enables spatial mapping of cellular structures and cellular structures in relation to the analytes. Cellular component barcoding can also serve as a reference marker, outlines the boundaries of a cell in relation to other cells, enables estimation of the size and shape of the cell, enables the measurement of the distance between a component and analyte etc.

The method can be used to map an epigenomics state. Examples include, but are not limited to, methylation, open-chromatin state (e.g., ATAC-seq), DNA-protein binding etc. Analytes can be pre-processed before barcode synthesis. For example, DNA can be modified with transposon sequences using a process called transposition (e.g. transposases) enabling ATAC-seq, cut-and-tag assays, whole-genome etc. Transposon sequences can include barcode(s), an UMI, a common sequence(s), a primer and can be part of the barcode synthesis module. The analyte of interest can be DNA, RNA, cDNA, protein, carbohydrate, small molecule, large molecule, drug, or any combination of analytes etc. In some embodiments, the transposition is performed under conditions that maintains cellular and DNA integrity e.g. DNA is only fragmented once the transposase is removed. Examples include, but not limited, contiguity-preserving transposition and tagmentation. Tn5 transposition used to modify DNA with adaptor and/or index sequences while preserving contiguity.

The sequencing data may be used to construct an image of the sample in which each barcode essentially becomes a pixel in the image. In some embodiments, the resolution of the image may be down to 1 nm, 10 nm, 100 nm, 1 um, or 10 um.

In these embodiments, the resulting image can be false colored, where the different colors correspond to different RNAs or epitopes, and the intensity of any color in any single pixel of a cell correlates with the number of sequence reads obtained for the analyte (for example through unique molecular identifiers (UMI) attached to the sequencing library or analyte). In many cases, the image may be superimposed with an image of the sample, stained as described above.

The methods described herein find general use in a wide variety of applications for analysis of any sample (e.g., in the analysis of tissue sections, sheets of cells, spun-down cells, etc.). Further, the method has a variety of clinical applications, including, but not limited to, diagnostics, prognostics, disease stratification, personalized medicine, clinical trials and drug accompanying tests.

In particular embodiments, the sample may be a section of any tissue, including skin (melanomas, carcinomas, etc.), soft tissue, bone, breast, colon, liver, kidney, adrenal, gastrointestinal, pancreatic, gall bladder, salivary gland, cervical, ovary, uterus, testis, prostate, lung, thymus, thyroid, parathyroid, pituitary (adenomas, etc.), brain, spinal cord, ocular, nerve, and skeletal muscle, etc. In some embodiments, the sample may be a tissue biopsy obtained from a patient. Biopsies of interest include both tumor and non-neoplastic biopsies of any tissue.

The above-described method can be used to analyze cells from a subject to determine, for example, whether the cell is normal or not or to determine whether the cells are responding to a treatment. In one embodiment, the method may be employed to determine the degree of dysplasia in cancer cells. In these embodiments, the cells may be a sample from a multicellular organism. A biological sample may be isolated from an individual, e.g., from a soft tissue. In particular cases, the method may be used to identify cancer cells in a sample.

In some embodiments, the method may involve obtaining data (an image) as described above (an electronic form of which may have been forwarded from a remote location), and the image may be analyzed by a doctor or other medical professional to determine whether a patient has abnormal cells (e.g., cancerous cells) or which type of abnormal cells are present. The image may be used as a diagnostic to determine whether the subject has a disease or condition, e.g., a cancer. In certain embodiments, the method may be used to determine the stage of a cancer, to identify metastasized cells, or to monitor a patient's response to a treatment, for example.

The compositions and methods described herein can be used to diagnose a patient with a disease. In some cases, the presence or absence of a biomarker in the patient's sample can indicate that the patient has a particular disease (e.g., a cancer). In some cases, a patient can be diagnosed with a disease by comparing a sample from the patient with a sample from a healthy control. In this example, a level of a biomarker, relative to the control, can be measured. A difference in the level of a biomarker in the patient's sample relative to the control can be indicative of disease. In some cases, one or more biomarkers are analyzed in order to diagnose a patient with a disease. The compositions and methods of the disclosure are particularly suited for identifying the presence or absence of, or determining expression levels, of a plurality of biomarkers in a sample.

In some cases, the compositions and methods herein can be used to determine a treatment plan for a patient. The presence or absence of a biomarker may indicate that a patient is responsive to or refractory to a particular therapy. For example, a presence or absence of one or more biomarkers may indicate that a disease is refractory to a specific therapy, and an alternative therapy can be administered. In some cases, a patient is currently receiving the therapy and the presence or absence of one or more biomarkers may indicate that the therapy is no longer effective.

In some cases, the method may be employed in a variety of diagnostic, drug discovery, and research applications that include, but are not limited to, diagnosis or monitoring of a disease or condition (where the image identifies a marker for the disease or condition), discovery of drug targets (where a marker in the image may be targeted for drug therapy), drug screening (where the effects of a drug are monitored by a marker shown in the image), determining drug susceptibility (where drug susceptibility is associated with a marker) and basic research (where is it desirable to measure the differences between cells in a sample).

In certain embodiments, two different samples may be compared using the above methods. The different samples may be composed of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen, etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material contains cells that are susceptible to infection by a pathogen such as a virus, e.g., human immunodeficiency virus (HIV), etc., and the control material contains cells that are resistant to infection by the pathogen. In another embodiment, the sample pair is represented by undifferentiated cells, e.g., stem cells, and differentiated cells.

The images produced by the method may be viewed side-by-side or, in some embodiments, the images may be superimposed or combined. In some cases, the images may be in color, where the colors used in the images may correspond to the sequences of the nucleic acids.

Cells from any organism, e.g., from bacteria, yeast, plants and animals, such as fish, birds, reptiles, amphibians and mammals may be used in the subject methods. In certain embodiments, mammalian cells, i.e., cells from mice, rabbits, primates, or humans, or cultured derivatives thereof, may be used.

Also provided by this disclosure are kits for practicing the subject methods, as described above. In some embodiments, the kit may comprise, e.g., an enzyme mix for barcode synthesis, selected from a polymerase and a terminal transferase, a reversible terminator nucleotide, and one or more oligonucleotides for barcode synthesis.

The various components of the kit may be present in separate containers or certain compatible components may be precombined into a single container, as desired.

In addition to the above-mentioned components, the subject kit may further include instructions for using the components of the kit to practice the subject method.

Also provided is a system, where the system may comprise the components of the kit as well as a spatially addressable deprotection system that is capable of applying an external stimulus, e.g., a light stimulus, an electrochemical stimulus or a pH change to areas on a substrate. The system may comprises a mask, a digital microarray mirror, optical projection, or patterned electrodes, for example, as well as another other components, e.g., a light source, or electrical source, for performing the method.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Human vs Mouse Incorporation of A vs U

In this example, barcode synthesis is demonstrated in situ with Nuclei using a universal template (5 nitroindole base). Cellular genomic DNA is modified with adapters using contiguity-preserving tagmentation in the open chromatin state of the cell. The adapters are subsequently modified through extension using a therminator nucleotide. After incorporation of the therminator the sample is exposed to UV light (de-blocking) to allow further extension and library preparation. In this example, ATAC-seq libraries are only prepared if UV light is used. Sequencing results confirm that the Therminator nucleotide is incorporated over the 5′ nitroindole universal base, the A therminator nucleotide specifically incorporated for Mouse Cells, and the U Nucleotide therminator incorporated for the Human cells. This demonstrated that cellular samples can be barcoded with some of the methods and compositions described in this application.

The protocol used is described below:

Oligos: Extended Transposon 5P_sp8_A14_ME
/5Phos/CGCGTCGC
TCGTCGGCAGCGTCAGATGTGTATAAGAGACAG
Adaptor (extension) SBS318_SBS521
ACGACGCTCTTCCGATCT
GAGTTCTACAGTCCGACGATC
Extended Splint-5N A14′sp8′_1N_SR521′-ex6
GACGCTGCCGACGA
GCGACGCG/i5NitInd/GATCGTCGGACTGTAGAACTC
AGATCG/3C6/

(1) Cell/Nuclei Prep:

(2) Collect human cells (K562) and mouse cells (3T3)

(3) 1× Wash of cells with PBS

(4) spin down and resuspend 500 uL ice cold NIB 0.02% NP40

(5) Incubate for 10 min on ice

(6) Spin down @ 500 g for 3 min

(7) Wash with PBS+BSA

(8) Resuspend tubes in 300 ul of PBS+BSA

(9) Quant nuclei using Trypan blue on countess, Nuclei nuclei/uL

Tagmentation:

(10) Make 1 million cells for each cell type:
Reagent Volume (uL) MM×2
nuclei 5 10 uL
500 nM TSM 5 10 uL
ETB3 5 10
Incubate at 55 C for 15 min
Add 200 uL TMG buffer, pulse vortex to mix
Spin 500×g for 5 min
Remove supernatant leaving 20 uL
Splint Hyb:
Add 5 uL 2 uM A14′sp8′_1N_SR521′-ex6/SBS318_SBS521 duplexes (annealed: 1× annealing buffer annealrt program)
Distribute into 4 tubes by 5 uL/ea
Incubate 55 C 5 min, then place on ice
Extension:
Make the following mastermix:

| | Stock | | Final | | Volume | |
|---|---|---|---|---|---|---|
| Reagent | conc | | conc | | (uL) | MM ×9 |
| H2O | 2.25 | | 20.25 | | | |
| 10× Ther. Buffer | 10× | | 1× | | 1 | 9 |
| photo A or photo U | 2 | mM | 0.25 | mM | 1.25 | add indiv |
| or 0.1× TE | | | | | | |
| Therminator or SDB | 2 | U/uL | 1 | U | 0.5 | add indiv |
| Tgmted nuclei + oligos | | | | | 5 | add indiv |

| | |
|---|---|
| Total volume | 10 |

Add 3.25 uL mastermix to each reaction
Add 1.25 uL photo A or photo U or 0.1×TE
Add 0.5 uL Therminator or SDB and pulse vortex to mix
Incubate 55 C for 15 min
UV Deblock:
Expose tubes 10V 20 sec
Ligation:
Add 10 uL 2× ligase buffer
Add 0.5 uL T3 ligase to each sample
Incubate RT for 45 min
Zymo/SPRI Stop Tagmentation
To all samples add 1× (21 uL) DNA binding buffer from zymo DNA clean & concentrator kit (cat no D4004)
Vortex to mix
Incubate 5 min RT
Add 1×SPRI (42 uL)
Vortex to mix
RT 5 min
Wash 2× with 200 uL 80% EtOH
Elute in 12 uL RSB
Store excess sample not used in PCR at 4 C
Veraseq Ultra PCR:
Veraseq Ultra Master mix: Make 1 per 500 primer

| Reagent | Stock | Final | 1× vol | ×20 MM1 | ×9 MM2 |
|---|---|---|---|---|---|
| Veraseq ultra | 2× | 1× | 6.25 | 125.0 | 83.8 |
| H2O | | 3.06 | 61.3 | | |
| A501 or N501 | 100 uM | 500 nM | 0.06 | Add in MM2 | 0.6 |
| N70X | 10 uM | 500 nM | 0.63 | add indiv | add indiv |
| Template | | | 2.50 | add indiv | add indiv |
| Total vol | | | 12.50 | 186.25 | |

Distribute mastermix by 9.4 ul
Add 0.63 individual N70x's primers
Add 2.5 ul sample
Run the following PCR program:
72 C 5 min
98 C 30 sec
98 C 10 sec 15 cycles
66 C 30 sec
72 C 30 sec
72 C 1 min
10 C forever
Ran 2 uL on a 1.2% Lonza gel with Promega 100 bp ladder
Clean and Nextseq: (full length only)
Add 1×SPRI (10 uL)
Vortex to mix
RT 5 min
Wash 2× with 200 uL 80% EtOH
Elute in 12 uL RSB
Store excess sample not used in PCR at 4 C
Dilution: 0.3 nM

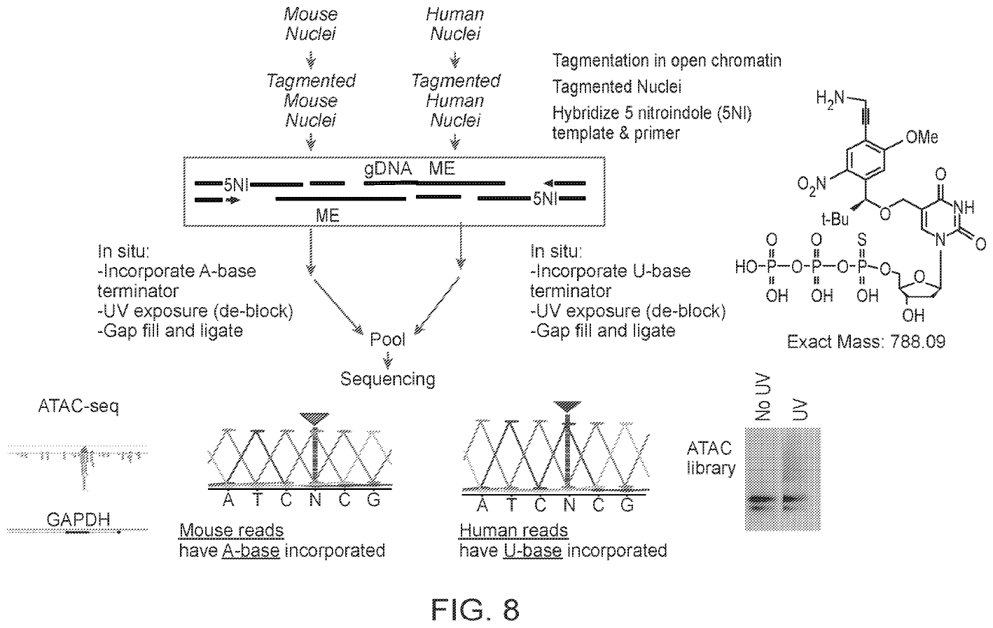

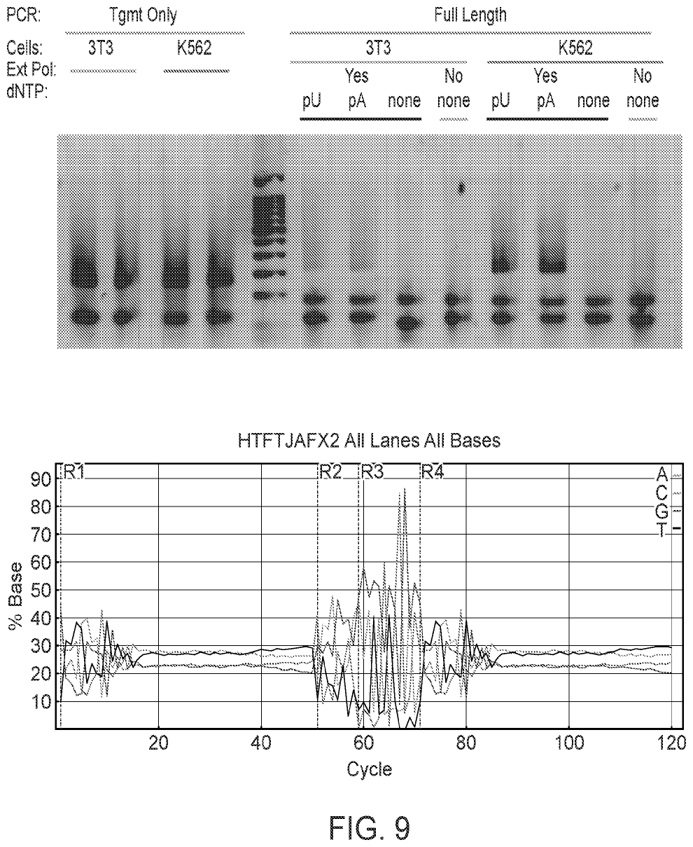

The strategy used for these experiments is illustrated in FIG. 8. Results are shown in FIG. 9. A summary of the conditions are described in the table below.

| | | | Ext | Full | | |
|---|---|---|---|---|---|---|
| | | | poly- | Length | | ~nM |
| Condition | Nuclei | dNTP | merase | index | ng/uL | (400 bp) |
| 1 | Mouse | Photo U | Yes | N707 | 4.06 | 15.4 |
| 2 | | Photo A | Yes | N708 | 4.18 | 15.8 |
| 3 | | none | Yes | N709 | 1.01 | 3.8 |
| 4 | | none | No | N710 | 1.57 | 5.9 |
| 5 | Human | Photo U | Yes | N711 | 6.6 | 25 |
| 6 | | Photo A | Yes | N712 | 6.42 | 24.3 |
| 7 | | none | Yes | N701 | 1.64 | 6.2 |
| 8 | | none | No | N704 | 1.84 | 7 |

1. A method for synthesizing spatially addressed nucleic acid barcodes in or on a cellular sample in situ, comprising:
(a) obtaining a cellular sample comprising nucleic acid molecules that are protected by a reversible terminator;
(b) deprotecting the nucleic acid molecules in a set of areas of the sample by selectively applying an external stimulus to the set of areas to produce deprotected nucleic acid molecules in the areas;
(c) applying a reversible terminator nucleotide to the cellular sample, resulting in addition of a reversible terminator onto the deprotected nucleic acid molecules;
(d) optionally removing any unreacted reversible terminator nucleotide after step (c); and
(e) repeating steps (b)-(d) one or more times, to produce spatially addressed barcodes that are attached to nucleic acid molecules that are in or on the cellular sample.

2. The method of claim 1, further comprising sequencing the barcodes produced in step (e) and at least part of the nucleic acid molecules to which they are attached, or an amplification product thereof.

3. The method of claim 2, further comprising mapping the sequenced nucleic acid molecules to a site in or on the cellular sample using the barcode to which it is attached.

4. The method of claim 1, wherein the cellular sample is a tissue section.

5. The method of claim 1, wherein the cellular sample of (a) is obtained by hybridizing, ligating or binding an oligonucleotide that is protected by a reversible terminator or can be protected to a sample that contains cells.

6. The method of claim 1, wherein the cellular sample of (a) is obtained by reversibly terminating nucleic acid molecules that are native to the sample.

7. The method of claim 1, wherein the cellular sample of (a) is made by:
(i) blocking the 3′ hydroxyls that are present in nucleic acids that are endogenous to the sample, or
(ii) binding an oligonucleotide that is protected at the 3′ end by the reversible terminator to the sample or is blocked after binding,

8. The method of claim 7, wherein the oligonucleotide is bound to nucleic acid in the sample by hybridization.

9. The method of claim 7, wherein the oligonucleotide is tethered to a binding agent (e.g., an antibody or aptamer) that is bound to a protein in or on the sample or the oligonucleotide is part of binding agent complex.

10. The method of claim 1, wherein the addition of step (c) is templated.

11. The method of claim 1, wherein the addition of step (c) is non-templated.

12. The method of claim 1, wherein the nucleic acid molecules of (a) are protected by a reversible terminator at the 3′ end, and the addition of step (c) is an addition to the 3′ end of the deprotected nucleic acid molecules.

13. The method of claim 12, wherein the addition of step (c) is done enzymatically.

14. The method of claim 13, wherein the addition of step (c) is non-templated and catalyzed by a terminal transferase,

15. The method of claim 13, wherein the addition of step (c) is templated and catalyzed by a polymerase.

16. The method of claim 1, wherein the nucleic acid molecules of (a) are protected by a reversible terminator at the 5′ end, and the addition of step (c) is an addition the 5′ end of the deprotected nucleic acid molecules.

17. The method of claim 16, wherein the cellular sample of (a) is made by binding oligonucleotides that are protected at the 5′ end by a reversible terminator to the sample.

18. The method of claim 16, wherein the addition of step (c) is done using phosphoramidite or H-phosphonate addition chemistry.

19. The method of claim 1, wherein the set of areas of (b) comprises at least 10, at least 100 (e.g., at least 1,000, at least 5,000, at least 10,000, at least 50,000, at least 100,000, at least 500,000, or at least 1M) areas.

20. The method of claim 1, wherein the external stimulus applied in (b) is a light stimulus, an electrochemical stimulus or a pH change.

21. The method of claim 1, wherein the external stimulus is selectively applied by a mask, a digital microarray mirror, optical scanner, optical projection, or patterned electrodes.

22. The method of claim 1, wherein steps (b)-(d) are repeated at least 2 times.

23. The method of claim 1, wherein the barcodes produced in step (e) are at least 4 nucleotides in length.

24. The method of claim 1, wherein the reversible terminator nucleotide added in one or more of the repeats is different to the reversible terminator nucleotide in a prior repeat.

25. The method of claim 1, wherein the cellular sample of (a) is made by:
i. hybridizing a tailed reverse transcription primer to RNA in the cellular sample;
ii. extending the reverse transcription primer in situ to produce extension products that comprise the sequence of the tailed reverse transcription primer and first strand cDNA; and
iii. hybridizing a splint oligonucleotide and a primer to the extension products in situ, wherein the splint oligonucleotide comprises internal universal nucleotides and a primer has a 3′ reversible terminator, and wherein the extension products, splint oligonucleotide and the primer hybridize to produce a complex in that contains a gap between the 5′ end of the cDNA and the 3′ end of the primer, wherein the gap is across from the universal nucleotides.

26. The method of claim 25, wherein the barcode is made by (i) adding nucleotides to the 3′ end of the primer across from the universal nucleotides to make an extension product and (ii) sealing the extension product to the 5′ end of the extension products by ligation.

27. The method of claim 25, wherein the primer is oligo(dT), a random primer or target-specific primer.

28. The method of claim 1, wherein the reversible terminator of step (a) or at least one of the reversible terminators added in step (c) comprises an affinity tag.

29. The method of claim 5, wherein the oligonucleotide comprises an affinity tag.

30. The method of claim 1, wherein at least some of the repeats of (e) the set of areas that are deprotected in step (b) is different to but overlapping with the prior set of areas that are deprotected.

31-32. (canceled)
